(19) United States
(12) Patent Application Publication
(10) Pub. No.: US 2009/0286383 A1
(43) Pub. Date: Nov. 19, 2009

(54) TREATMENT OF WHISKERS

(75) Inventors: Nan Jiang, Austin, TX (US); Zvi Yaniv, Austin, TX (US)

Correspondence Address: FISH & RICHARDSON P.C., P.O BOX 1022, Minneapolis, MN, 55440-1022, US

(73) Assignee: APPLIED NANOTECH HOLDINGS, INC., Austin, TX (US)

(21) Appl. No.: 12/466,306

(22) Filed: May 14, 2009

Related U.S. Application Data
(60) Provisional application No. 61/099,027, filed on Sep. 22, 2008.
(60) Provisional application No. 61/053,574, filed on May 15, 2008.

(51) Int. Cl. H01L 21/20; B05D 3/06; H01L 21/26; A61N 5/00

(52) U.S. Cl. 438/478; 427/553; 438/795; 250/492.2; 977/896; 977/901; 977/762; 257/E21.328; 257/E21.09

(57) ABSTRACT

A photo-curing or photosintering process is utilized to modify, reduce or eliminate whiskers or nanowires growing on a material surface.

This application claims priority to U.S. Provisional Application Serial Nos. 61/099,027 and 61/053,574, which are hereby incorporated by reference herein.

### TECHNICAL FIELD

The present invention relates in general to nano-sized wires and whiskers.

### BRIEF DESCRIPTION OF THE DRAWINGS

FIG. 1 illustrates a flow chart of fabrication of nanowires/whiskers and photosintering of these nanowires/whiskers.

FIG. 2 shows a digital image of CuO nanowires/whiskers having been grown on a Cu wire substrate by a thermal oxidation method.

FIGS. 3A and 3B show digital images of how a photo-curing treatment effectively changes the CuO nanowire morphology.

FIGS. 4A and 4B illustrate a system and method for in accordance with an embodiment of the present invention.

FIGS. 5A and 5B show digital images of optical microscopy (OM) photographs taken from a tin whisker sample before (FIG. 5A) and after (FIG. 5B) photosintering.

FIG. 6 shows a digital image of a tin whisker without photosintering.

FIGS. 7A and 7B show digital images of SEM images of a photosinter-treated whisker.

### DETAILED DESCRIPTION

For decades Sn/Pb components and circuit assemblies have been used for high reliability, high performance military, space and commercial applications. With government legislation intended to eliminate lead from electronics manufacturing and packaging, the next generation of high performance devices will likely rely on lead-free technology. Unfortunately, lead-free surfaces readily form nanowires or whiskers that can grow in length may cause electrical short circuits between adjacent components, circuit elements, or devices. The tendency of tin surfaces to form nanowires or whiskers is prevalent, in particular, during the electro-deposition processes utilized in the electronic industry. One way of mitigating the nanowire or whisker formation is to realize a more robust tin-based electro-deposition process with very low potential for whisker growth, which eventually can be used to fabricate electronic circuits in high reliability weapon systems. One approach to improve electro-deposition may require control of a large number of parameters such as dopants, impurities, additives, and wetting agents. An alternative approach is to develop post-deposition or even post-assembly treatments to the circuit board assemblies to eliminate the nanowires or whiskers and/or limit their formation.

The assignee of this application has been successful in developing a copper conductive ink using copper metallic nanoparticles for inkjet printed electronic applications on various substrates (see U.S. Provisional Applications Ser. Nos. 61/077,711, 61/081,539 and 61/053,574, all of which are hereby incorporated by reference herein). The inkjet deposition process for this copper ink is compatible with ambient atmospheric environments (no inert environments are required), and a drying process may be performed at temperatures under 100° C. This achievement results from a process of photosintering the printed copper ink traces, whereby a very short flash of photonic energy transforms the dried copper ink to metallic copper traces with resistivities as low as 3×10⁻⁶ ohm-cm.

The aforementioned photosintering process may also be utilized to flatten (melt down or evaporate away) copper nanowires and copper whiskers to the level of the base copper surface, basically causing them to disappear. This process can also be applied to reduce or eliminate tin whiskers or nanowires that are developing during the electro-deposition process by utilizing the photosintering processes and systems as post deposition and/or post assembly processes. This process will also work to eliminate or at least diminish tin whiskers or nanowires as well as or better than as demonstrated on copper whiskers due to tin's lower melting temperature. Furthermore, embodiments of the present invention can be used to diminish or eliminate nanowires or whiskers of other materials. The photosintering systems are inexpensive, are commercially available, and require less than 1 second process time. Furthermore, they can be comfortably integrated with current laboratory and industrial electro-deposition systems.

Treatment, elimination, diminishment, reduction of nanowires and whiskers:

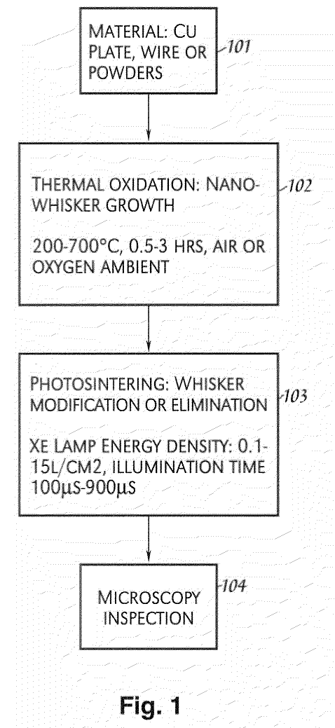

Referring to FIG. 1, there is illustrated a flow chart of fabrication of CuO nanowires/whiskers and photosintering of these nanowires/whiskers. Or, the same process can be performed on Tin (Sn).

In step 101, provided is a copper substrate, such as a substrate with copper electroplated thereon, a copper wire on a substrate, or copper powders on a substrate. In step 102, the copper oxide nanowires/whiskers grow as a result of thermal oxidation. The growth parameters may be 200-700° C. for 0.5-3 hours in air or ambient oxygen environment. The nanowires/whiskers are then diminished, reduced in number and/or length, or eliminated with photosintering in step 103. Photosintering may be performed with an Xₑ lamp at an energy density of 0.1-15 J/cm² for 100-900 μs. In step 104, the sample may then be inspected by microscopy.

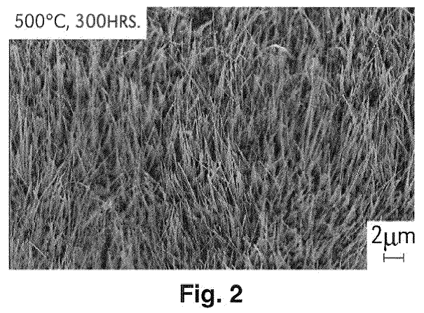

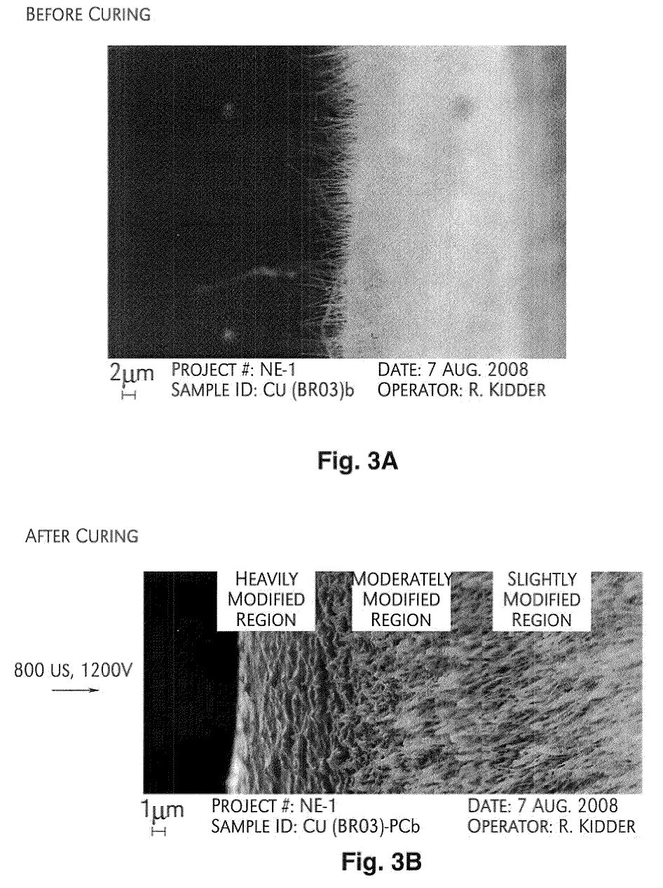

The digital photo of FIG. 2 shows CuO nanowires/whiskers having been grown on a Cu wire substrate by a thermal oxidation method. The CuO nanowires may be mass produced by oxidation of μm-sized Cu particles (powders). The digital photos of FIGS. 3A, 3B show how a photo-curing treatment effectively changes the CuO nanowire morphology. The photo-curing (photosintering) technique is also able to bond the CuO nanowires on polymer substrates by exposing the CuO nanowires through a transparent substrate, exposing from the transparent side. Combining the photo-curing (photosintering) and CuO nanowire fabrication techniques together leads to useful applications in sensor, solar cell or flexible devices. By reducing the intensity of the photocuring (photosintering) process, the effect on the CuO nanowires (or, Sn nanowires or whiskers) can be reduced or modified to achieve other effects.

CuO nanowires are a promising p-type semiconductor material (1.2-1.4 eV band gap). CuO nanowires have potential applications as gas sensors and as counter electrodes of dye solar cells. Combined with the photosintering technique, the CuO nanowires may become much more useful. For example, photosintering can bond the CuO nanowires on polymer substrates for producing flexible devices. Photosintering may also modify the CuO nanowires (or other metal or metal oxide nanowires or whiskers) to meet application requirements.

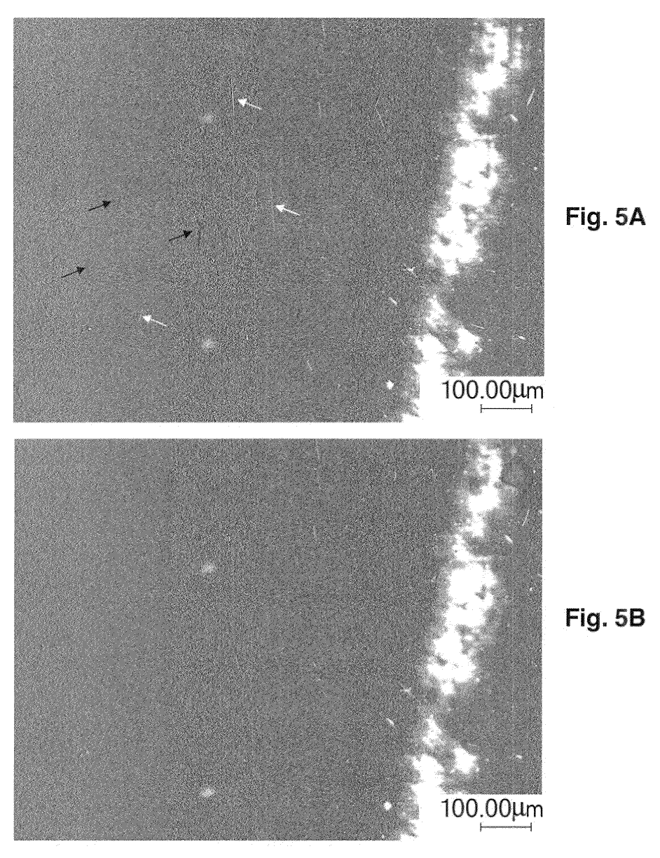

FIGS. 5A and 5B show digital images of optical microscopy (OM) photographs taken from a tin whisker sample before (FIG. 5A) and after (FIG. 5B) photosintering. The photosintering parameters are: 1600 V discharge voltage with a 500 μs pulse width. Since the photographs are reflective OM images, the tin whiskers lying on the sample surface present a bright contrast (as indicated by white arrows in FIG. 5A), and those running out the surface present the dark contrast (as indicated by black arrows in FIG. 5B). Note that the broad bright band shown in the right portion of FIGS. 5A, 5B is merely a man-made scratch as a reference point for assisting in finding the same area before and after photosintering. FIGS. 5A, 5B show that after photosintering a portion of the tin whiskers disappeared, and the remainder of the tin whisker became shorter. Moreover, photosintering acts to more likely destroy the tin whiskers running out of the surface. The pulse photosintering operated at the current condition is approximately estimated to be able to eliminate more than 70 vol/% of the tin wishers.

The mechanism accounting for the huge change of tin whiskers after pulse photosintering is the fast thermal effects. The heat generated in tin whiskers during the intense photo flashing causes the tin whiskers to plastically distort, melt, or even evaporate, since the tin has relatively low melting and boiling points. This is strongly supported by SEM observation of the samples.

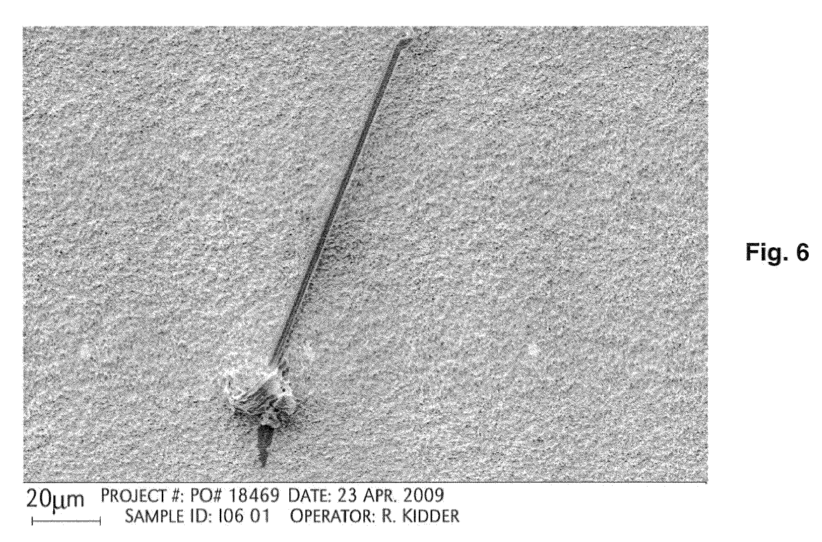

The digital image in FIG. 6 shows a tin whisker without photosintering. It is straight and presents “perfect” surface facets.

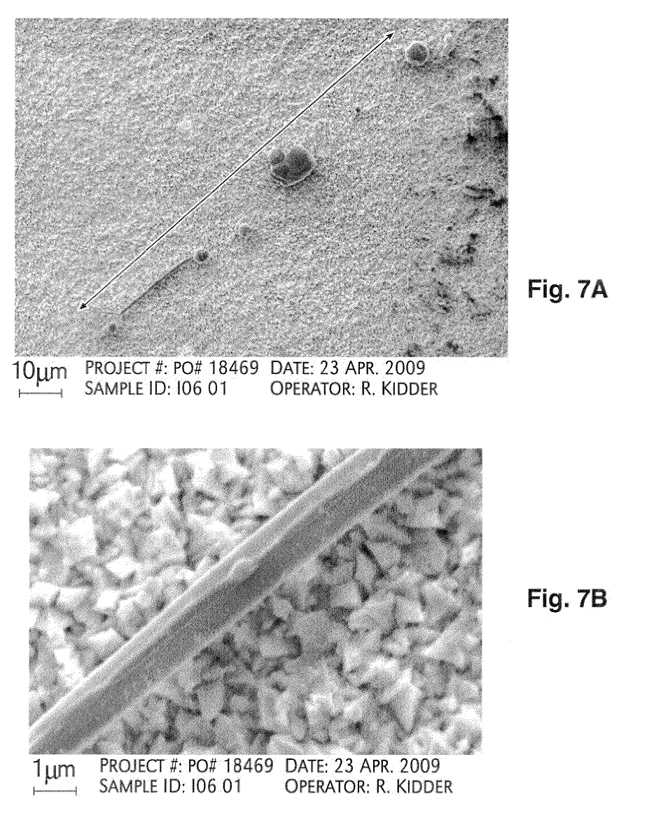

The digital images in FIGS. 7A and 7B show SEM images of a photosinter-treated whisker, which has a very different morphology to the untreated one in FIG. 6. The whisker in FIG. 7A was partly melted and shortened. A few “small balls” of the tin whisker are located on the two ends of the shown tin whisker and along the tin whisker extension line. The balls were formed from the tin molten liquid; the original tin whisker was at least more than 3 times longer, as indicated by the white arrow in FIG. 7A. When the tin whisker was melted, the molten liquid physically constrains into droplets due to surface tension, leaving the metal balls on the sample surface along the original tin whisker line. FIG. 7B reveals distinct melted surface features.

The SEM digital images in FIGS. 6, 7A, and 7B show that the thermal effect works on the tin whiskers during photosintering. Since it is a thermal process, it is time and energy dependent. Thus far, experiments reveal that a higher voltage/shorter pulse discharge is more efficient.

The thermal effect nature causes the treatment efficiency to depend on the heat dissipative ways. The tin whiskers running out of or normal to a substrate surface are more likely to be destroyed than those lying on the substrate surface, because the latter more easily dissipate the heat flow to the substrate, as is show in FIGS. 5A, 5B. Since most tin whiskers that cause electronic short-circuits are those running out of or normal to a substrate surface, the photosintering methods of the present invention are effective for alleviating this problem.

Energy flash curing involves a short, high intensity light pulse. This light is absorbed by the metal nanoparticles and converted to heat. This heat then causes the metal nanowires or whiskers to deform and melt. A high intensity lamp may be used to supply enough energy. Energy flash curing is most effective when it is used in the high intensity—short time flash mode, because under these conditions the thermal energy that is produced remains within the nanowires or whiskers, and there is minimal or no thermal damage to the substrate surface. A shorter flash time provides sufficient energy to penetrate the metal plating surface and relieve the stress and strain of the plated material without compromising the surface film.

The optical energy that is absorbed by whiskers or nanowires will convert into thermal energy (DE). This thermal energy will increase the temperature of the whiskers or nanowires. Some thermal energy from the nanomaterials will dissipate into the substrate and the surrounding air. For bulk metal, the increased temperature (DT) can be given by the equation: ΔE=mC, ΔT, where m is the mass of bulk metal, and Cp is the specific heat of the metal. Because the energy flash occurs in the microsecond time scale, the tin does not have the time to oxidize, thus maximizing the electronic conductivity. In oven curing, for example, the curing process can take hours, which allows the sample to oxidize if air is allowed to be present.

The maximum temperature reached by the substrate or other parts on the substrate is less than 100° C. This low curing temperature prevents the substrate or electronic components from thermal breakdown that would otherwise occur at higher temperatures demanded by other deposition processes. This low temperature process is non-destructive. The broad spectrum source is non-damaging even to photo-sensitive materials such as Kevlar or optical top coats.

Embodiments of the present invention will successfully operate to diminish or eliminate metal whiskers (e.g., Sn or Ni whiskers), oxide whiskers (e.g., CuO, or ZnO), or compound and element semiconductor whiskers (e.g., GaN or Si whiskers).

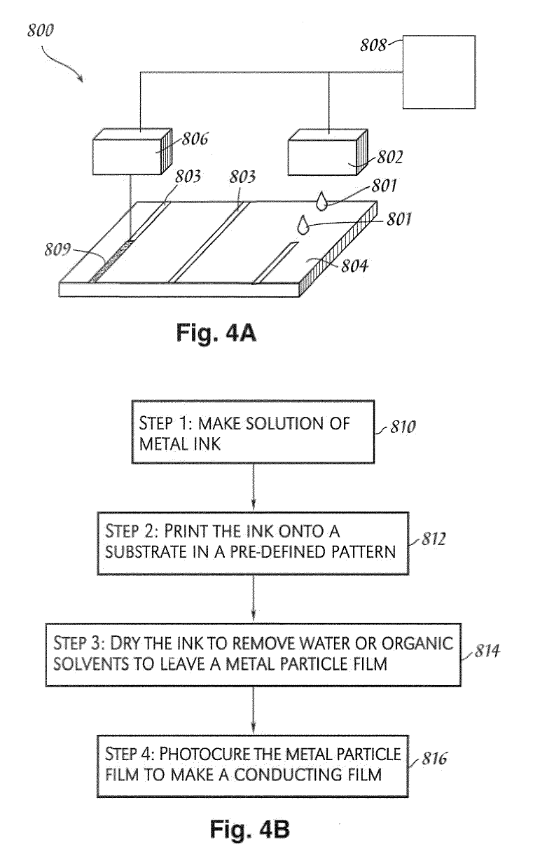

Referring to FIG. 4A, a system 800 is illustrated for deposition (e.g., inkjetting) and photo-curing of nanoparticle metal films (e.g., copper or any other suitable metal ink). The system 800 includes an inkjet dispenser 802 for dispensing metal ink 801 onto the surface of a substrate 804. The system 800 also includes a light source 806 for curing the ink films 803 deposited by the inkjet dispenser 802. The light source 806 may be a laser source (pulsed or continuous), a pulsed lamp, or a focused beam, such as described herein. In some implementations, the dispenser 802 is arranged to automatically pass over the substrate along a predetermined pathway 803. Additionally, the dispenser 802 may be arranged to dispense the metal ink 801 at multiple predetermined positions and times above the substrate 804. The light source 806 may be attached to the inkjet dispenser 802 or arranged to travel over the substrate 804 separately from the dispenser 802. The light source 806 may be arranged to photo-cure the inkjetted films 803 immediately after they are deposited by the dispenser 802. Alternatively, the light source 806 may be arranged to photo-cure the films 803 at predetermined times following the deposition of the film. The motion of the light source 806 and the dispenser 802 may be controlled by a computer system/controller arrangement 808. A user may program the computer 808 such that the controller automatically translates the dispenser 802 and light source 806 over a predetermined path 803. In some implementations, the light source 806 and dispenser 802 are fixed, and the substrate 804 is placed on a movable platform (not shown) controlled by the computer/controller 808.

A flow chart of a photo-curing process is illustrated in FIG. 4B. In step 810, a solution of metal ink 801 is mixed. In step 812, the ink 801 is printed or dispensed onto the substrate 804 using the dispenser 802. The film 803 deposition may be tightly controlled so a well-defined pattern 803 is formed. For example, the metal film patterns 803 may be closely aligned conductive circuitry, which can then experience short-circuits due to the growth of nanowires/whiskers on the films 803. The film is then dried in step 814 to eliminate water or solvents.

In some embodiments, a thermal curing step can be introduced subsequent to dispensing the film 803 and prior to the photo-curing step 816. The substrate 804 and deposited film 803 may be cured using an oven or by placing the substrate 804 on the surface of a heater, such as a hot plate. For example, in some implementations, the film 803 is pre-cured in air at 100° C. for 30 minutes before photo-curing. Alternatively, the thermal curing may be performed by directing a laser onto the surface of the film 803. Following the drying and/or thermal curing step 814, a laser beam or focused light from the light source 806 may be directed in step 816 onto the surface of the film 803. The light source may serve to photo-cure the film 803 such that it has low resistivity. Generally, the metal films are insulating after the printing/dispensing 812 and drying 814 steps. After the photo-curing process, however, the insulating film becomes a conductive film 809 (see FIG. 4A). Additionally or alternatively, the light source 806 may serve to modify, diminish, or eliminate nanowires or whiskers from the film 803.

In some implementations, the dispenser 802 is used to deposit a blanket film or a coarse outline of the pattern. Typically, printing techniques can achieve feature sizes on the order of 25-50 microns or greater. If finer features are necessary, the pattern/blanket film can be refined or reduced using a focused beam of light or laser, in which case the features are defined by the spot size of the laser or by the focus of the light beam. Typically, light can be focused to 1 micron or less. Thus, submicron features may be possible. Ultimately, the feature size may be limited by the size of the nanoparticles used in the conductive film. Metal particles may be formed to have features on the order of 1-5 nm.

What is claimed is:

1. A method comprising:
growing nanowires or whiskers on a substrate; and
photosintering the nanowires or whiskers.

2. The method as recited in claim 1, wherein the nanowires or whiskers are grown by thermal oxidation.

3. The method as recited in claim 1, wherein the nanowires or whiskers comprise copper.

4. The method as recited in claim 1, wherein the nanowires or whiskers comprise tin.

5. The method as recited in claim 1, wherein the substrate is produced by electro-deposition.

6. The method as recited in claim 1, wherein the nanowires or whiskers comprise a metal material.

7. The method as recited in claim 1, wherein the nanowires or whiskers comprise oxide nanowires or whiskers.

8. The method as recited in claim 1, wherein the nanowires or whiskers comprise compound and element semiconductor nanowires or whiskers.

9. The method as recited in claim 1, wherein the whiskers comprise micron-sized whiskers.

10. The method as recited in claim 1, wherein the photosintering is performed with a Xenon lamp having an energy range from 0.1-15 J/cm² with a treatment time between 20-900 microseconds.

11. A method for reducing or eliminating whiskers by photosintering the whiskers.

12. The method as recited in claim 11, wherein the whiskers are grown by thermal oxidation.

13. The method as recited in claim 11, wherein the whiskers comprise copper.

14. The method as recited in claim 11, wherein whiskers comprise tin.

15. The method as recited in claim 11, wherein the whiskers comprise a metal material.

16. The method as recited in claim 11, wherein the whiskers comprise oxide whiskers.

17. The method as recited in claim 11, wherein the whiskers comprise semiconductor whiskers.

18. The method as recited in claim 11, wherein the whiskers comprise micron-sized whiskers.

19. The method as recited in claim 11, wherein the whiskers comprise nano-sized whiskers.

20. The method as recited in claim 11, wherein the photosintering is performed with a Xenon lamp having an energy range from 0.1-15 J/cm² with a treatment time between 20-900 microseconds.
